(19) United States
(12) Patent Application Publication
(10) Pub. No.: US 2007/0020752 A1
(43) Pub. Date: Jan. 25, 2007

(54) Cell culturing device and system

(75) Inventors: Rosalind Russell, Lower Sackville (CA); Judy Angelbeck, Huntington, NY (US)

Correspondence Address: LEYDIG VOIT & MAYER, LTD, 700 THIRTEENTH ST. NW, SUITE 300, WASHINGTON, DC, 20005-3960, US

(73) Assignee: Pall Corporation, East Hills, NY (US)

(21) Appl. No.: 10/557,198

(22) PCT Filed: May 18, 2004
(86) PCT No.: PCT/US04/15668

§ 371 Date: Nov. 17, 2005

Related U.S. Application Data
(60) Provisional application No. 60/472,425, filed on May 22, 2003.

(51) Int. Cl. C12M 1/12

(52) U.S. Cl. 435/297.5; 435/304.1

(57) ABSTRACT

Devices, systems, and methods for culturing cells and providing a contaminant-free connection are disclosed. A cell culture device comprises a container having first and second side walls wherein the first and second side walls are gas permeable, the device further comprising a female connector and a male connector. At least one of the connectors can be coupled to the corresponding connector of another cell culture device while providing a contaminant-free connection.

### CROSS-REFERENCE TO RELATED PATENT APPLICATIONS

This patent application claims the benefit of U.S. Provisional Patent Application No. 60/472,425, filed May 22, 2003, which is incorporated by reference.

### FIELD OF THE INVENTION

This invention relates to a device, system, and method for culturing cells.

### BACKGROUND OF THE INVENTION

A variety of systems exist for culturing cells, e.g., so that the number of cells can be expanded, directed to undergo differentiation and/or so that cells can be cultured to produce a soluble substance for harvest.

However, conventional systems have suffered from a number of drawbacks. For example, some cells are maintained in rigid, gas impermeable plastic culture flasks wherein the lids or caps of the flasks are loosened to allow gas exchange with the outside environment, e.g., too provide oxygen to serve the aerobic needs of the cells, and carbon dioxide to maintain the pH of the growth medium. This can allow contaminants to enter the flasks, adversely affecting the cells and/or the desired cell product. Moreover, since culturing the cells can include transferring cells from one flask to another (e.g., cells can be transferred from a source flask to a transfer flask for sub-culturing), the transfer includes placing the source and transfer flasks under a laminar flow hood, removing the caps from the flasks, using a pipette to transfer the cells from one flask to another, and replacing the caps. This can also introduce contamination into either or both flasks or containers, and can detrimentally affect the cells and/or products.

Cells can also be maintained in gas permeable bags having side walls wherein each side wall is formed from multiple films or a film with multiple layers. However, some bags have been unsuitable for growing the cells for a desired period of time. Additionally, transferring cells from one bag to another typically includes placing the source and transfer bags under a laminar flow hood, and using a needle or spike connected to a conduit that is in fluid communication with the transfer bag, wherein the needle or spike is briefly uncovered before inserting it into a septum on the source bag. The cells are then transferred through the conduit from one bag to another. This connection (e.g., including briefly leaving the needle open to the atmosphere) can also introduce contamination into either or both bags, and can detrimentally affect the cells and/or products.

Moreover, transferring cells from one flask to another or one bag to another as described above requires skilled lab personnel and can be labor intensive.

Accordingly, there is a need in the art for a container and system that is suitable for culturing cells while minimizing the potential for contamination during cell processing and/or cell transfer.

The present invention provides for ameliorating at least some of the disadvantages of the prior art. These and other advantages of the present invention will be apparent from the description as set forth below.

### BRIEF SUMMARY OF THE INVENTION

In accordance with an embodiment of the invention, a device for use in cell culture systems is-provided, comprising a container and at least one fitting, the container being manufactured from a gas permeable film and having first and second side walls, an internal volume, and at least first and second fluid flow ports, the container being suitable for containing a cell culturing medium and/or cells to be cultured therein, wherein the first and second side walls are permeable to gas; the fitting being in fluid communication with the internal volume of the container, the fitting being couplable to an additional fitting for use in providing a contaminant-free connection, the additional fitting being in fluid communication with an additional container.

In an embodiment of the device, the device includes an additional (second) fitting, the second fitting in fluid communication with the internal volume of the container, the second fitting being couplable to an additional fitting for use in providing a contaminant-free connection, the additional fitting being in fluid communication with an additional container.

In a typical embodiment, the device includes a conduit connected to a fitting and a fluid flow port. For example, in an embodiment of the device including first and second fittings, a first conduit is connected to the first fluid flow port and the first fitting, and a second conduit is connected to the second fluid flow port and the second fitting.

In one embodiment, the container is manufactured from a gas permeable film comprising a copolymer comprising ethylene and an acrylate, typically, ethylene and an alkyl acrylate. In a more preferred embodiment, the gas permeable film comprises a copolymer comprising ethylene and an alkyl acrylate comprising butyl acrylate or methyl acrylate, even more preferably, wherein the walls of the container comprise a gas permeable polymeric film manufactured from a copolymer comprising ethylene and at least 20 wt. % butyl acrylate or at least 20 wt. % methyl acrylate.

In another embodiment, the container is manufactured from a gas permeable film comprising a polyvinyl chloride compound, the polyvinyl chloride compound comprising a medium molecular weight polyvinyl chloride resin and a plasticizer, or an ultra high molecular weight polyvinyl chloride resin and a plasticizer.

In another embodiment, a cell culture system is provided, the system comprising at least two cell culture devices as described above. Accordingly, a fitting from one device is couplable to a fitting from another device, wherein the fittings, once coupled together, provide a contaminant-free connection, and fluid can be transferred from one device to another.

In preferred embodiments of the cell culture device and cell culture system, at least one fitting, more preferably, each fitting, defines an aperture, and the fittings each include a removable sealing layer, more preferably a removable contamination containment sealing layer, sealing the aperture.

For example, in an embodiment of the cell culture system including first and second cell culture devices, the first device including a first couplable fitting, and the second device including a second couplable fitting, the system includes a connector assembly including the couplable fittings, wherein the assembly comprises the first fitting, the first fitting defining a first aperture, the first fitting including a first removable sealing layer sealing the first aperture, and the additional (second) fitting, the second fitting defining a second aperture, the second fitting including a second removable sealing layer sealing the second aperture, more preferably, wherein the first fitting includes a stem member mounted in the fitting, the stem member having a head axially movable into the aperture of the second fitting. The assembly can include a resilient sealing member positioned between the first and second fittings and communicating with the first and second apertures. More preferably, each fitting includes a resilient sealing member coupled to the respective aperture. In accordance with an embodiment of the invention, the fittings of the connector assembly are coupled together, the first and second contamination containment layers are removed, the stem member is moved axially so that the head moves into the aperture of the second fitting and a fluid flow path is established through the first and second fittings while providing a contaminant-free connection.

In a more preferred embodiment, the cell culture system includes first and second cell culture devices, each cell culture device including first and second couplable fittings, wherein the first fitting of the first cell culture device is couplable to the second fitting of the second cell culture device, and the second fitting of the first cell culture device is couplable to the first fitting of the second cell culture device, each first and second fitting defining an aperture, and including a removable sealing layer sealing the aperture, each first fitting of one culture device preferably including a stem member mounted in the fitting, the stem member having a head axially movable into the aperture of the second fitting of the other cell culture device. In accordance with an embodiment of the invention, the first fitting of one device is coupled to the second fitting of the other device, the first and second sealing layers are removed, the stem member is moved axially so that the head moves into the aperture of the second fitting and a fluid flow path from one device to the other device is established through the first and second fittings while providing a contaminant-free connection.

Preferably, cell culture systems according to embodiments of the invention allow cells to be maintained in culture and sub-cultured for several weeks, or more, while minimizing the potential for introducing contamination into the cell culture.

### BRIEF DESCRIPTION OF THE DRAWINGS

FIG. 1 shows an embodiment of a cell culture device according to the invention.

FIG. 2 shows an embodiment of a cell culture system according to the invention, including two cell culture devices and first and second connectors connectable to form a connector assembly.

FIG. 3 is a side view of a connector assembly according to an embodiment of the present invention.

FIG. 4 is a side view in partial section of the connector assembly of FIG. 3.

FIG. 5 is a side view in partial section of the connector assembly with the stripout layers removed and the stem inserted into the female fitting.

FIG. 6 is an end view of a male connector in an unconnected state.

FIG. 7 is an end view of a female connector in an unconnected state.

FIG. 8 is a sectional view of a male sealing member.

FIG. 9 is a sectional view of a female sealing member.

FIG. 10a is a side view in partial section of the male connector cap.

FIG. 10b is a side view in partial section of the female connector cap.

FIG. 10c is a top view of the male or female connector cap.

FIG. 11 shows an illustrative arrangement for transferring fluid to or from a cell culture device according to the invention, wherein a manifold having a plurality of connectors allows a connector to be coupled to the cell culture device.

### DETAILED DESCRIPTION OF THE INVENTION

In accordance with an embodiment of the present invention, a device for cell culture is provided comprising a container manufactured from a polymer film, the container having an internal volume and at least first and second fluid flow ports, and having first and second side walls permeable to gas, and, at least a first fitting, the first fitting being in fluid communication with the internal volume of the container, the fitting being couplable to an additional fitting for use in providing a contaminant-free connection, the additional fitting being in fluid communication with an additional container. In some embodiments, the device includes cell culturing medium in the container.

In an embodiment of the device, the device also includes second fitting, the second fitting in fluid communication with the internal volume of the container, the second fitting being couplable to an additional fitting for use in providing a contaminant-free connection, the additional fitting being in fluid communication with an additional container.

In one embodiment, the container is manufactured from a gas permeable film comprising a copolymer comprising ethylene and an acrylate, typically, ethylene and an alkyl acrylate. In a more preferred embodiment, the gas permeable film comprises a copolymer comprising ethylene and an alkyl acrylate comprising butyl acrylate or methyl acrylate, even more preferably, wherein the walls of the container comprise a gas permeable polymeric film manufactured from a copolymer comprising ethylene and at least 20 wt. % butyl acrylate or comprising ethylene and at least 20 wt. % methyl acrylate.

In another embodiment, the container is manufactured from a gas permeable film comprising a polyvinyl chloride compound, the polyvinyl chloride compound comprising a medium molecular weight polyvinyl chloride resin and a plasticizer, or an ultra high molecular weight polyvinyl chloride resin and a plasticizer.

Typically, the gas permeable walls allow oxygen transmission into the interior of the container, and carbon dioxide transmission from the interior of the container to the exterior of the container. The gas permeability is sufficient for cell respiration, and this permeability can be desirable for various cell metabolic functions during the culture period. Preferably, the gas permeability is selected to optimize cell growth.

In some embodiments, the film forming the side walls of the container has a 22° C. room air oxygen transmission of about 12 μmoles or greater O₂/hr/350 cm² film surface area, preferably, a 22° C. room air oxygen transmission of about 15 μmoles or greater O₂/hr/350 cm² film surface area, and even more preferably, a 22° C. room air oxygen transmission of about 20 μmoles or greater O₂/hr/350 cm² film surface area.

In another embodiment, a cell culture system is provided, the system comprising at least two cell culture devices as described above. Accordingly, at least one fitting from one device is couplable to at least one fitting from another device, wherein the fittings, once coupled together, provide a contaminant-free connection.

In preferred embodiments of the device and system, at least one fitting, more preferably, each fitting, defines an aperture, and the fittings each include a removable sealing layer, more preferably a removable bacteria blocking sealing layer, sealing the aperture.

For example, in an embodiment of the cell culture system including at least first and second cell culture devices, the first device including a first couplable fitting, and the second device including a second couplable fitting, the system includes a connector assembly including the couplable fittings, wherein the assembly comprises the first fitting, the first fitting defining a first aperture, the first fitting including a first removable bacteria blocking sealing layer sealing the first aperture, and the additional (second) fitting, the second fitting defining a second aperture, the second fitting including a second removable bacteria blocking sealing layer sealing the second aperture, more preferably, wherein the first fitting includes a stem member mounted in the fitting, the stem member having a head axially movable into the aperture of the second fitting. The assembly can include a resilient sealing member positioned between the first and second fittings and communicating with the first and second apertures. More preferably, each fitting includes a resilient sealing member coupled to the respective aperture. In accordance with a preferred embodiment of the invention, the fittings of the connector assembly are coupled together, the first and second bacteria blocking sealing layers are removed, the stem member is moved axially so that the head moves into the aperture of the second fitting and a fluid flow path is established through the first and second fittings while providing a contaminant-free connection.

In a more preferred embodiment, the cell culture system includes at least first and second cell culture devices, each cell culture device including first and second couplable fittings, wherein the first fitting of the first cell culture device is couplable to the second fitting of the second cell culture device, and the second fitting of the first cell culture device is couplable to the first fitting of the second cell culture device, each first and second fitting defining an aperture, and including a removable bacteria blocking sealing layer sealing the aperture, each first fitting of one culture device including a stem member mounted in the fitting, the stem member having a head axially movable into the aperture of the second fitting of the other cell culture device. In accordance with a preferred embodiment of the invention, the first fitting of one device is coupled to the second fitting of the other device, the first and second bacterial blocking sealing layers are removed, the stem member is moved axially so that the head moves into the aperture of the second fitting and a fluid flow path from one device to the other device is established through the first and second fittings while providing a contaminant-free connection.

In accordance with embodiments of a system according to the invention including connector assemblies, a connector assembly provides a contaminant-free connection to maintain the sterility of a fluid passing through the connector assembly. Preferred connector assemblies include those disclosed in U.S. Pat. Nos. 5,810,309 and 6,536,805, as well as International Publication Nos. WO9630076, WO9408173, and WO 9850105, which are each incorporated by reference.

Embodiments of the invention are especially suitable for maintaining cells at high densities, e.g., allowing proliferation indices (viable cell concentration/initial viable cell concentration) of about 2×10⁶/ml or more, for example, about 3×10⁶/ml or more, or about 4×10⁶/ml or more.

Cells can be cultured in cell culture devices according to embodiments of the invention for any desired period of time. In some embodiments, cells are cultured for a few hours, e.g., wherein the cells are stimulated or activated (for example, with a soluble product such as interleukin). Typically, cells are cultured in cell culture devices according to embodiments of the invention for at least about two days, more typically, at least four days. In some embodiments, cells can be cultured in the inventive cell culture devices for at least about 6 days. Some types of cells can be cultured in cell culture systems according to embodiments of the invention for at least two weeks, or more.

A variety of cells can be processed, preferably cultured, in accordance with the invention, and embodiments of the invention comply with current Good Manufacturing Practices. Culturing can include maintaining, differentiating, expanding and/or propagating the cells in vitro. A variety of cells can be cultured in accordance with the invention, e.g., eukaryotic cells. While a variety of cells can be cultured in accordance with the invention, tumor cells, tumor-derived cells, and blood cells, especially human blood cells, including for example, lymphocytes, monocytes, neutrophils, neutrophil precursors, and immature blood cells including stem and progenitor cells are especially suitable. In one embodiment, the cells to be cultured are apheresis-derived cells.

Embodiments of the invention are suitable for processing adherent cells (sometimes referred to as anchorage-dependent cells) and/or non-adherent cells (sometimes referred to as suspension cells, or anchorage-independent cells).

A variety of cell culture media can be used in accordance with the invention, and the selection of suitable media and modifications thereof are known to one of skill in the art.

Embodiments of the invention are especially suitable for providing aseptic connections, more preferably, without requiring the use of a flow hood while making the connection and passing fluid from one cell culture device to another. Advantageously, an aseptic connection can be made, and fluid can be transferred, at a location without a flow hood, e.g., at a lab bench. In accordance with the invention, a fluid flow path, e.g., a liquid flow path, can be established, through the coupled fittings, allowing fluid to flow from one cell culturing device to another, wherein the fluid flow path is isolated from the ambient environment and from contaminants present in the ambient environment. In some embodiments, the fluid flow path can be established while maintaining a sterile fluid pathway, making the invention suitable for use in closed systems.

Each of the components of the invention will now be described-in more detail below, wherein like components have like reference numbers.

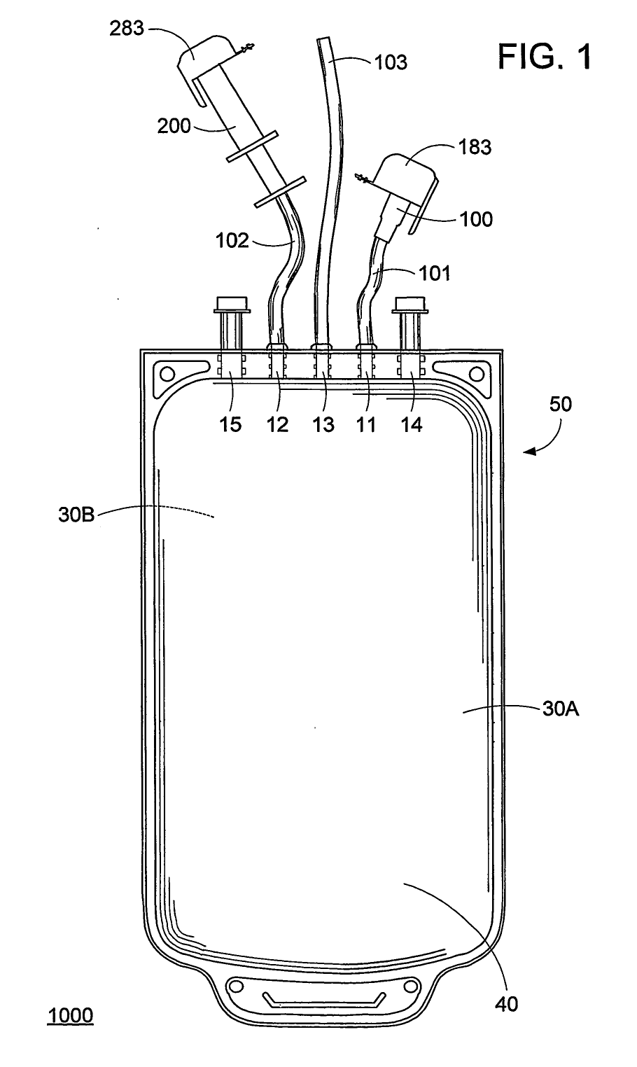

FIG. 1 illustrates an embodiment of a cell culture device 1000 comprising a container 50 (a flexible bag) manufactured from a polymeric film, the container having first and second side walls 30A and 30B, wherein the first and second side walls are gas permeable and are edge-sealed together. The first and second side walls each have an inner and an outer surface, wherein the inner surface is suitable for contacting the liquid contents of the container. The container 50 has an interior volume 40 suitable for containing fluid, preferably, cell- and/or cell culture-containing fluid, therein. The container has at least two fluid flow ports, and in the illustrated embodiment of the cell culture device, the container has five fluid flow ports, i.e., first fluid flow port 11, second fluid flow port 12, third fluid flow port 13, fourth fluid flow port 14, and fifth fluid flow port 15. The illustrated cell culture device 1000 includes first and second connectors, preferably female connector 100 and male connector 200.

Each connector can comprise any structure suitable for fluid communication. Each connector can be attached to or formed as part of any suitable fluid container or conduit, for example, a section of tubing, or a flexible container such as a bag. In the illustrated embodiment of the device, the first connector 100 is attached to one end of a first conduit 101, wherein the other end of the conduit is attached to the first fluid flow port 11 such that the first connector is in fluid communication with the interior volume of the container, and the second connector 200 is attached to one end of a second conduit 102, wherein the other end of the conduit is attached to the second fluid flow port 12 such that the second connector is in fluid communication with the interior volume of the container.

The first and second connectors are described in more detail below with respect to embodiments of the system, e.g., wherein a connector of one cell culture device is coupled to a connector of another cell culture device.

The illustrated embodiment of the cell culture device also includes a third conduit 103, wherein one end of the third conduit is attached to the third flow port 13, and the other end of the conduit is sealed, e.g., to allow for sterile docking to the conduit. The illustrated embodiment of the device also includes fourth fluid flow port 14 and fifth fluid flow port 15, that can be access ports, e.g., spike entry ports. In some embodiments (not shown), a luer connector and/or a needle-less connector, can be attached to at least one port and/or at least one conduit.

In other embodiments of one or more cell culture devices (not shown), the devices include a different number of at least one of ports, conduits, and connectors. For example, a cell culture device can include two or more ports, and a first connector or a second connector. Typically, the device includes a conduit attached to a fluid flow port and a connector (e.g., a first connector or a second connector).

The cell culture device 1000 (with or without cell culture media therein) can be sterilized as is known in the art, e.g., via steam, ethylene oxide (ETOH), or gamma, sterilization.

In one embodiment, the container 50 is manufactured from a gas permeable film comprising a copolymer comprising ethylene and an acrylate, typically, ethylene and an alkyl acrylate. In a more preferred embodiment, the gas permeable film comprises a copolymer comprising ethylene and an alkyl acrylate comprising butyl acrylate or methyl acrylate, even more preferably, wherein the walls of the container comprise a gas permeable polymeric film manufactured from a copolymer comprising ethylene and at least 20 wt. % butyl acrylate or comprising ethylene and at least 20 wt. % methyl acrylate.

In another embodiment, the container 50 is manufactured from a gas permeable film comprising a polyvinyl chloride compound, the polyvinyl chloride compound comprising a medium molecular weight polyvinyl chloride resin and a plasticizer, or an ultra high molecular weight polyvinyl chloride resin and a plasticizer.

Typically, the gas permeable walls 30A, 30B allow oxygen transmission into the interior of the container, and carbon dioxide transmission from the interior of the container to the exterior of the container. The gas permeability is sufficient for cell respiration, and this permeability can be desirable for various cell metabolic functions during the culture period. Preferably, the gas permeability is selected to optimize cell growth.

In some embodiments, the film forming the side walls of the container(s) has a 22° C. room air oxygen transmission of about 12 μmoles or greater O₂/hr/350 cm² film surface area, preferably, a 22° C. room air oxygen transmission of about 15 μmoles or greater O₂/hr/350 cm² film surface area, and even more preferably, a 22° C. room air oxygen transmission of about 20 μmoles or greater O₂/hr/350 cm² film surface area.

A variety of gas permeable films can be used to manufacture containers suitable for use in the invention. Suitable films and containers include, but are not limited to, those disclosed in U.S. Pat. Nos. 4,280,497, and 5,721,024, as well as in International Publication No; WO 02/065976.

Typically, embodiments of containers comprising a polymeric film manufactured from a copolymer comprising ethylene and an alkyl acrylate produced in accordance with the invention are free of, or essentially free of, plasticizers such as di (2-ethylhexyl) phthalate (DEHP), tri (2-ethylhexyl) trimellitate (TOTM), and citrate ester plasticizers such as n-butryl tri-n-hexyl citrate (BTHC). However, the containers (e.g., the polymeric film) can include modifiers and/or additives such as, for example, at least one of an antistatic, antiblock, a stabilizer, and antioxidant, e.g., for use in processing the film or resin (described below).

Typically, a resin is used in producing the polymeric film comprises at least one copolymer comprising ethylene and an acrylate, preferably comprising ethylene and an alkyl acrylate. The resin can comprise a plurality of copolymers, e.g., a blend comprising a first copolymer comprising ethylene and a first alkyl acrylate, and a second copolymer comprising ethylene and a second alkyl acrylate.

In some embodiments, the copolymer comprises ethylene and at least about 18 weight percent alkyl acrylate, typically, ethylene and at least about 20 weight percent alkyl acrylate, based upon the combined weight of the ethylene and the alkyl acrylate. For example, the copolymer can comprise ethylene and at least about 22 weight percent alkyl acrylate, or ethylene and at least about 24 weight percent alkyl acrylate. The term “alkyl” herein refers to an alkyl group having from 1 to about 10 carbon atoms, preferably from 1 to about 6 carbon atoms, and more preferably from 1 to about 4 carbon atoms. In even more preferred embodiments, the alkyl acrylate is methyl acrylate or butyl acrylate. For example, the resin can comprise a copolymer comprising ethylene, and at least about 18 wt. % methyl acrylate or at least about 18 wt. % butyl acrylate, preferably, ethylene, and at least about 20 wt. % methyl acrylate or at least about 20 wt; % butyl acrylate. In other embodiments, the resin comprises a copolymer comprising ethylene, and at least about 22 wt. % methyl acrylate or at least about 22 wt. % butyl acrylate, or ethylene and at least about 24 wt. % methyl acrylate or at least about 24 wt. % butyl acrylate.

Typically, the resin has a melt index of about 3 g or less per 10 min (in some embodiments, about 1 g or less) as measured by ASTM D 1238, condition 190° C./2.16 kg, and has a Vicat softening temperature (e.g., as measured by ASTM D 1525) of at least about 50° C.

Such resins are commercially available, e.g., from Eastman Chemical Company (Kingsport, Tenn.), Atofina Chemicals, Inc. (Philadelphia, Pa.) and Dupont (Wilmington, Del.). For example, a variety of resins commercially available from Eastman Chemical Company referred to as EMAC® (including EMAC+®), EBAC® (including EBAC+®), and EMAC/EBAC® are suitable. Illustrative examples of such resins are ethylene butyl acrylate copolymer (EBAC) resin, e.g., EBAC SP1802 and SP1903 specialty copolymers, and ethylene methyl acrylate copolymer (EMAC) resin, e.g., EMAC SP1305, SP1307, SP1330, SP1400, SP2202, SP2207, SP2220, SP2260 and SP2268, specialty copolymers. Illustrative suitable resins commercially available from Atofina Chemicals, Inc., include, for example, those resins referred to as LOTRYL™ resins (e.g., LOTRYL™ EBA and LOTRYL™ EMA) and illustrative suitable resins commercially available from DuPont include, for example, those resins referred to as ELVALOY™ resins (e.g., ELVALOY™ AC).

In another embodiment, the container 50 comprises a polyvinyl chloride film manufactured from a polyvinyl chloride compound, the polyvinyl chloride compound comprising an ultra high molecular weight polyvinyl chloride resin having an inherent viscosity of at least about 1.25, as measured by ASTM D-1243; and at least about 43 weight percent of a plasticizer. In some embodiments, the ultra high molecular weight polyvinyl chloride resin has an inherent viscosity of at least about 1.50, as measured by ASTM D-1243. Typically, the inherent viscosity is in the range of from about 1.25 to about 2.00. Alternatively, or additionally, in some embodiments, the compound includes at least about 53 weight percent of the plasticizer, typically, in the range of from about 53 to about 57 weight percent of the plasticizer.

In another illustrative embodiment, the container 50 comprises a polyvinyl chloride film manufactured from a polyvinyl chloride compound, the polyvinyl chloride compound comprising a medium molecular weight polyvinyl chloride resin and at least about 30 weight percent of a plasticizer. Typically, the compound includes in the range of from about 30 to about 50 weight percent of the plasticizer.

Preferably, in those embodiments wherein the container comprises a polyvinyl chloride film manufactured from a polyvinyl chloride compound, the plasticizer is one from the group of plasticizers consisting of: tri (2-ethylhexyl) trimellitate (TOTM); di-(2-ethylhexyl) phthalate; acetyl tri-n-butyl citrate; n-butyryl tri-n-hexyl citrate; acetyl tri-n-octyl citrate; and acetyl tri-n-decyl citrate.

A variety of conduits are suitable for use in the invention. Typically, the conduits are formed from plasticized PVC for flexibility and strength. In some embodiments, the conduits are formed from the same materials used to form the containers.

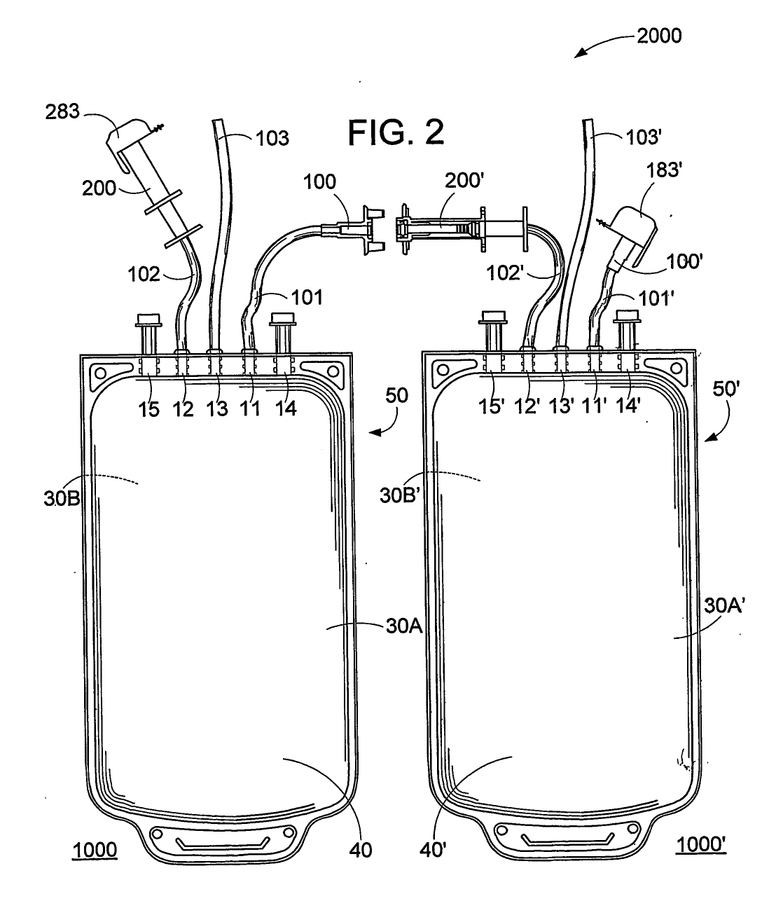

The containers and conduits used in the invention can have any suitable size, shape, internal volume and/or thickness. The containers and conduits can be made from the polymeric film and resin described herein using conventional techniques known and used in the industry. Illustratively, the bag can be arranged from a single sheet of sheet of film (e.g., folded over at the end where the ports are arranged and sealed around the other edges as shown in FIGS. 1 and 2), two sheets of film, from a collapsed blown bubble of film (sometimes referred to as “lay flat tubing”), and the like. The bags and conduits are typically extruded, but can be blow molded or formed by other appropriate methods known in the art.

Containers and conduits can be sealed as is known in the art, utilizing, for example, an adhesive, a solvent, radio frequency sealing, ultrasonic sealing and/or heat sealing. If desired, at least one port (or fitment) is formed using the polymers and/or copolymers described above, and/or by co-extruding other materials such as various polymeric materials. For example, at least one port (or any number of ports) can have an outer surface material of a copolymer comprising ethylene and an acrylate, and an inner surface material of polyvinyl chloride (PVC). Such a configuration can allow efficient formation of the seal between outer surface of the port and the bag body, and efficient formation of the seal between the inner surface of the port with a conduit comprising PVC.

The inner and/or outer surfaces of the container side walls 30A, 30B can be treated (e.g., to provide at least one of a coating, a chemical modification and a texture such as an embossment or etching) or, more typically, the surfaces can be untreated.

In typical embodiments of containers used in the invention, each side wall 30A, 30B is a single layer of film. An exemplary wall thickness of containers for cell culture fluids using the polymeric film can be, for example, in the range of about 0.005 to about 0.025 inch (about 0.13 to about 0.64 mm), in some embodiments, about 0.010 inch to about 0.018 inch (about 0.25 to about 0.46 mm), for example, about 0.012 to about 0.015 inch (about 0.30 to about 0.38 nm).

If desired, the container 50 can have sufficient tensile strength to withstand centrifugation. In some embodiments, the container 50 is resilient to temperature fluctuations, e.g., it can withstand low temperatures of for example, about −70° C. Advantageously, if desired, sterile media can be frozen in the container until the container is to be used.

Cells can be cultured in cell culture devices according to embodiments of the invention for a desired period of time. Preferably, however, the cells are transferred from one cell culture device to another, after making a contaminant-free connection between the devices, in accordance with embodiments of cell culture systems according to the invention.

FIG. 2 illustrates an embodiment of a cell culture system 2000 comprising first and second cell culture devices 1000, 1000′. In this illustrated embodiment, the first and second cell culture devices are essentially identical, although the invention is not so limited.

For example, as noted above, cell culture devices can differ with respect to the number of at least one of any of ports, conduits, and connectors. Alternatively, or additionally, in some embodiments of systems according to the invention, a plurality of containers can be made from the same, or different materials, depending on the particular application and/or cell culture system.

Moreover, while the illustrated embodiment of the system includes first and second culture devices, embodiments of the system ban include any number of culture devices, e.g., the number of culture devices can depend on, for example, how many times the cells are to be sub-cultured.

First cell culture device 1000 shown in FIG. 2 has been described above with respect to FIG. 1. The illustrated second culture device 1000′ comprises a container 50′ (a flexible bag) manufactured from a polymeric film, the container having first and second side walls 30A′ and 30B′, wherein the first and second side walls are gas permeable and are edge-sealed together. The container 50′ has an interior volume 40′ suitable for containing fluid, preferably, cell- and/or cell culture-containing fluid, therein. The container has at least two fluid flow ports, and in the illustrated embodiment of the cell culture device the container has five fluid flow ports, i.e., first fluid flow port 11′, second fluid flow port 12′, third fluid flow port 13′, fourth fluid flow port 14′, and fifth fluid flow port 15′.

In the illustrated embodiment of the system, each cell culture device includes first and second connectors. Alternatively, for example, one cell culture device can comprise a first connector, and another cell culture device can comprise a second connector. For convenience, the following discussion will typically refer to elements of the first and second cell culture devices, wherein the corresponding elements of the second cell culture devices are in parentheses.

The illustrated embodiment of each device in the system includes first and second connectors, preferably female connector 100 (100′) and male connector 200 (200′). Each connector can comprise any structure suitable for fluid communication. Each connector can be attached to or formed as part of any suitable fluid container or conduit, for example, a section of tubing, or a flexible container such as a bag. In the illustrated embodiments, the first connector 100 (100′) is attached to one end of a first conduit 101 (101′), wherein the other end of the conduit is attached to the first fluid flow port 11 (11′) such that the first connector is in fluid communication with the interior volume of the container, and the second connector 200 (200′) is attached to one end of a second conduit 102 (102′), wherein the other end of the conduit is attached to the second fluid flow port 12 (12′) such that the second connector is in fluid communication with the interior volume of the container.

The exemplary female connector 100 (100′) shown in FIGS. 1-3 generally comprises a fitting 120 (120′), preferably of unitary construction. The exemplary male connector 200 (200′) shown in FIGS. 1-3 generally comprises a stem 210 (210′) and a fitting 220 (220′). The fittings 120, 220 (120′, 220′) of the female and male connectors 100, 200 (100′, 200′) are preferably formed from a polymeric material. For example, the fittings 120, 220 (120′, 220′) may be molded from a polymeric material such as polycarbonate or polypropylene. For directional orientation in the following discussion, each connector has a proximal end, nearest the opposing connector, and a distal end furthest from the opposing connector. Also, since the exemplary connectors 100, 200 (100′, 200′) in FIG. 1 comprise generally elongated bodies, the term axial denotes disposition along their axes.

The female connector 100 of the first cell culture device 1000 is couplable to the male connector 200′ of the second cell culture device 1000′ (e.g., as shown in FIG. 2). Similarly, the male connector 200 of the first cell culture device 1000 is couplable to the female connector 100′ of the second cell culture device 1000′. This allows either set of male and female connectors to be coupled, as appropriate.

The female and male connectors 100, 200 (100′, 200′) may also comprise an interlocking mechanism adapted to interlock the male connector from one cell culture device in predetermined relation with the female connector from the other cell culture device.

The interlocking mechanism may have any suitable configuration, such as interlocking sleeves or threaded connections. In a preferred embodiment, e.g., as shown in FIGS. 1-3, and 7, the portion of the interlocking mechanism on the female fitting 120 (120′) includes a bracket 140 (140′). The bracket may be variously configured. The bracket 140 (140′) may comprise a socket 145 (145′) or cup having any suitable plan form, e.g., rectangular or circular. Typically, the bracket comprises a generally C-shaped member. The representative bracket may include a flange 142 (142′) and a generally cylindrical sidewall 144 (144′) defining a socket 145 (145′). The flange 142 (142′) may assume a radially extending annular plan form. In some embodiments, the sidewall 144 (144′) extends from and is concentric with the flange 142 (142′) and includes an annular proximal end surface 143 (143′) facing the male connector of the other cell culturing device.

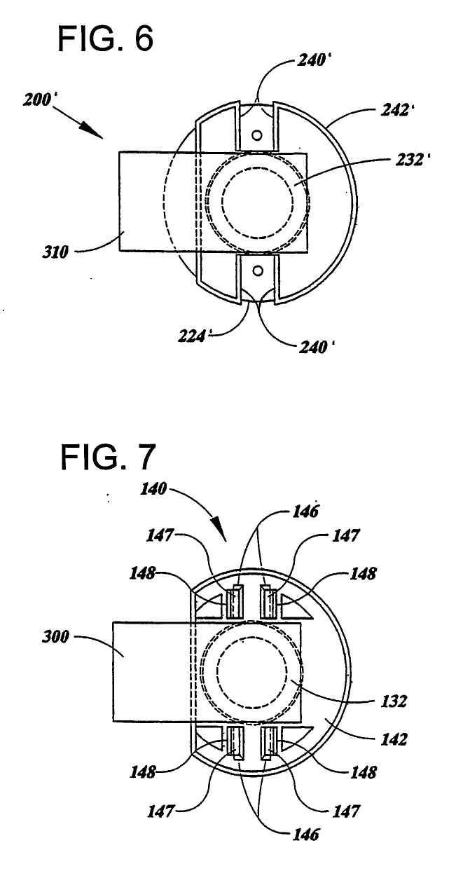

One or more forks 146 (146′) may extend from the flange 142 (142′). The forks 146 (146′) may be formed integrally with the flange 142 (142′). When the female connector from one device is coupled to the male connector of the other device, the forks preferably register in slots 240 (240′) (e.g., as shown in FIGS. 1 and 6) formed in an upper flange 242 (242′) of the male connector of the other device.

While in the illustrated embodiment, the forks 146 (146′) extend from a female connector and the slots 240 (240′) are in the male connector, the forks and slots may instead be associated with the male and female connectors, respectively. Each fork 146 (146′) in one device preferably comprises first and second prongs 147 (147′) which are preferably flexible to allow the prongs 147 (147′) to enter and lock in the slots 240 (240′) of the other device. Catches 148 (148′) can be formed on the prongs 147 (147′) of the forks 146 (146′) which pass through the slots 240 (240′) and abut a distal surface of the upper flange 242 (242′). In this manner, the forks 146 (146′) from one device extend through the slots 240 (240′) of the other device and engage the respective upper flange 242 (242′) of the male connector 200 (200′) to interlock the connectors 100 and 200′, or 100′ and 200.

The female connector 100 (100′) is preferably adapted to contain fluid and conduct fluid communication and preferably defines an isolated portion of the fluid flow path, e.g., containing or conducting isolated fluid communication. The female fitting 120 (120′) may define an internal chamber or aperture 132 (132′) which may have any suitable configuration and preferably has an open proximal end.

The distal end 126 (126′) of the female fitting 120 (120′) maybe connected to any suitable fluid container or, more preferably, bonded to or molded integrally with a first conduit 101 (101′). Similarly, the distal end 226 (226′) of the male fitting 220 (220′) may be connected to any suitable fluid container or, more preferably, bonded to or molded integrally with a second conduit 102 (102′). The first conduit 101 (101′) may be connected in fluid communication with the internal chamber 132 (132′) of the female fitting 120 (120′). The internal chamber 132 (132′) may comprise a bore 134 (134′) relieved at its proximal end into a counterbore 136 (136′) having a larger inner diameter than the bore 134 (134′). The cylindrical sidewall 144 (144′) surrounds the proximal end of the chamber 132 (132′) and defines the counterbore 136 (136′).

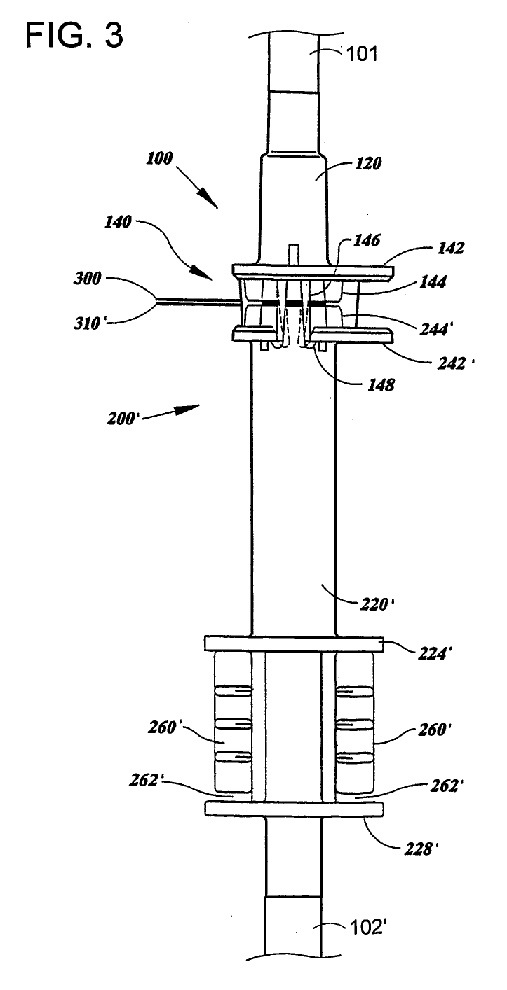

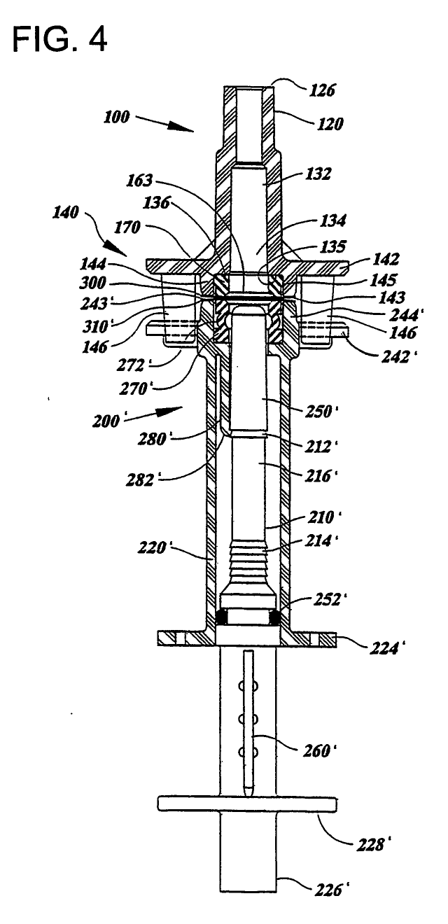

The female connector 100 (100′) preferably further comprises a sealing layer sealing the open proximal end of the aperture 132 (132′) in the female fitting 120 (120′). For example, the sealing layer preferably comprises a removable sealing layer, such as a female stripout layer 300 (300′) removably attached to the proximal end of the female fitting 120 (120′). In the embodiment illustrated in FIGS. 3 and 4, the female stripout layer 300 (300′) is attached to the open proximal end of the sidewall 144 (144′). For example, the female stripout layer 300 (300′) may be bonded to the proximal end surface 143 (143′) of the female fitting 120 (120′) through any suitable technique, for example, ultrasonic welding. The stripout sealing layer 300 (300′) preferably seals the chamber 132 (132′) of the female connector 100 (100′) from the ambient atmosphere. The female stripout sealing layer 300 (300′) preferably includes a pull tab that extends beyond the periphery of the connectors 100, 200 to allow removal when the connectors 100, 200 are joined.

The male connector 200 (200′) also preferably comprises a sealing layer which seals the open proximal end of an aperture 232 (232′) in the male fitting 220 (220′). For example, the sealing layer preferably comprises a removable sealing layer such as a male stripout layer 310 (310′) removably attached to the proximal end of the male fitting 220 (220′). In the embodiment illustrated in FIG. 4, the male stripout layer 310 (310′) is attached to the proximal end surface 243 (243′) at the open end of a generally cylindrical sidewall 244 (244′) at the proximal end of the male fitting 220 (220′). The inner and outer diameters of the male sidewall 244 (244′) may be approximately equal to those of the female sidewall 244 (244′). The male strip out sealing layer 310 (310′) may be bonded to the proximal end surface 243 (243′) of the male connector through any suitable technique, for example, ultrasonic welding. The male stripout sealing layer 310 (310′) preferably seals the interior of the male connector 200 (200′) from the ambient environment. The male stripout sealing layer 310 (310′) preferably includes a pull tab that extends beyond the periphery of the connectors 100 (100′), 200 (200′) to allow removal when the connectors are joined (e.g., connector 100 joined to connector 200′ or connector 100′ joined to connector 200).

When the female and male connectors from the two cell culture devices are initially connected, the female and male stripout sealing layers preferably abut one another in face-to-face contact.

Illustratively, when the female connector 100 of device 1000 and the male connector 200′ of device 1000′ are initially connected, the female and male stripout sealing layers 300, 310′ preferably abut one another in face-to-face contact. For example, the diameters and locations of the female and male sidewalls 144, 244′ and the lengths of the forks 146 and the sidewalls 144, 244′ may be arranged to provide face-to-face contact of the stripout layers 300, 310′ between the end surfaces 143, 243′ of the sidewalls 144, 244′ when the connectors 100 and 200′ are coupled. The dimensions may be arranged to provide not only contact but also a slight compression of the stripout layers 300, 310′ between the end surfaces 143, 243′. However, the compression is preferably not so large as to interfere with the removal of the stripout layers 300, 310′ from between the sidewalls 144, 244′. Of course, if the male and female connectors 100, 200′ include non-removable sealing layers, rather than the stripout sealing layers 300, 310′, then the compression may be somewhat larger. Alternatively, the dimensions and locations of the forks 146 and the sidewalls 144, 244′ may be arranged to provide a slight space between the female and male stripout layers 300, 310′. For example the combined length of the sidewalls 144, 244′ maybe less than the distance between the flanges 142, 242′. Preferably the space is sufficiently small to prevent significant axial movement of the connectors 100, 200′ when they are connected to one another.

The stripout layers 300 (300′), 310 (310′), may comprise impermeable materials, such as glassine paper, metal foils, or impermeable polymeric films, or permeable materials, including papers such as Tyvek™ paper or porous polymeric films, which preclude the passage of bacterial contaminants. A preferred impermeable material is an aluminum foil which is removably sealed to the fittings 120 (120′), 220 (220′). Permeable or porous materials offer the advantage, if desired, of allowing sterilizing gases, including ethylene oxide gas, to penetrate therethrough and spread to the interior of the male and female connectors 100 (100′), 200 (200′), thereby sterilizing them without having to remove the stripout layers 300 (300′), 310 (310′). Either permeable or impermeable materials may be suitable for gamma or heat sterilization. Additionally, a bacteriostatic or bacteriocidal compound or layer (not illustrated) may be disposed on any, any combination, or all, stripout layers 300 (300′), 310 (310′). The female stripout layer(s) 300 (300′) maybe the same as or different from the male stripout layer(s) 310 (310′).

Although the illustrated embodiment depicts female and male connectors 100 (100′), 200 (200′) each with connecting ends sealed by removable sealing layers 300 (300′), 310 (310′), one, or each, or any combination of the connectors 100 (100′), 200 (200′) may additionally include a separate sealing layer, such as a pierceable membrane layer, which is not removable and is sealed to the connector under the stripout layer to provide an added level of sterility assurance. In other alternatives, the connectors 100 (100′), 200 (200′) may each include proximal ends sealed by sealing layers which are not removable, and the stripout layers may be omitted; or one connector may include only a stripout sealing layer while other connector(s) include only a non-removable sealing layer.

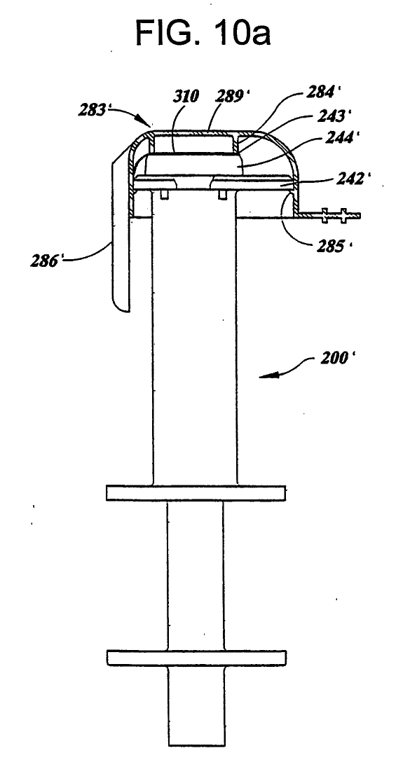

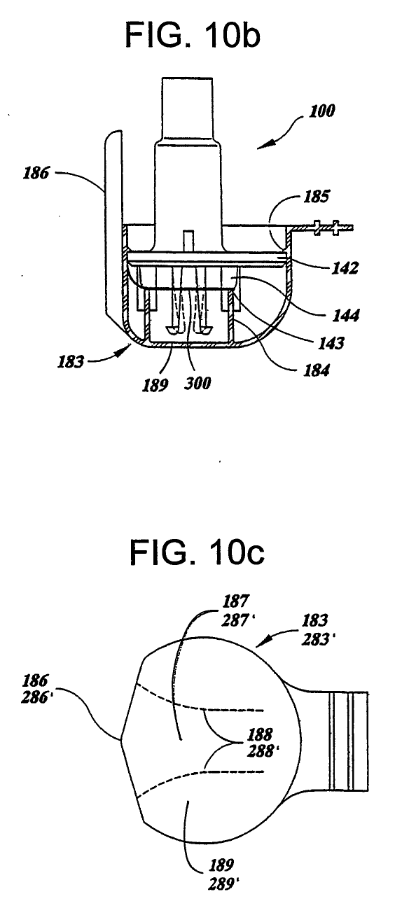

One, preferably each, of the connectors 100 (100′), 200 (200′) may also include a device which protects the proximal end of the connector and prevents the stripout layer 300 (300′), 310 (310′) from being inadvertently punctured or removed prior to assembly of the connectors (e.g., 100 to 200′, 100′ to 200). Preferably the device is operatively associated with the proximal ends of the connectors and can be easily removed prior to the assembly of the connectors. As shown in FIG. 10a and 10b, an exemplary embodiment of the device may be a cap 183 (183′), 283 (283′) which may include a cover 189 (189′), 289 (289′), a tab 186 (186′), 286 (286′) attached to the cover, a cylindrical sleeve 184 (184′), 284 (284′), and a plurality of ribs 185 (185′), 285 (285′).

Preferably the cover 189 (189′), 289 (289′) has a dome-shaped configuration, although a cover may have any other suitable configuration such as a cylindrical configuration. One of the ends of the sleeve 184 (184′), 284 (284′) is attached to the inner surface of the cover.

When the cap 183 (183′), 283 (283′) is mounted to the proximal end of the connector 100 (100′), 200 (200′), the other end of the sleeve 184 (184′), 284 (284′) bears against the end 143 (143′), 243 (243′) of the sidewall 144 (144′), 244 (244′), and the ribs 185 (185′), 285 (285′) engage the flange 142 (142′), 242 (242′) of the connector 100 (100′), 200 (200′).

Thus, the sleeve 184 (184′), 284 (284′) and the ribs 185 (185′), 285 (285′) allow the cap 183 (183′), 283 (283′) to be securely mounted to the proximal end of the connector 100 (100′), 200 (200′). Further, the sleeve presses the stripout layer300 (300′), 310 (310′) against the end 143 (143′), 243 (243′) of the sidewall 144 (144′), 244 (244′), holding the stripout layer 300 (300′), 310 (310′) in place and preventing it from being torn off. Preferably the height of the cover 189 (189′), 289 (289′) and the length of the sleeve 184 (184′), 284 (284′) are chosen such that the parts at the proximal end of the connector 100 (100′), 200 (200′), such as the stripout layers 300 (300′), 310 (310′) and the forks 146 (146′), can be contained in and protected by the cap 183 (183′), 283 (283′).

Further, the tab 186 (186′), 286 (286′), which may be attached to the outer periphery of the cover 189 (189′), 289 (289′), preferably is sufficiently long such that the pulling tab 300 (300′), 310 (310′) are contained in and protected by the tab 186 (186′), 286 (286′).

To make a cap 183 (183′), 283 (283′) easily removable, the cap may include (e.g., as illustrated in FIG. 10c) a strip 187 (187′), 287 (287′) defined by perforations 188 (188′), 288 (288′) and connected to the tab 186 (186′), 286 (286′). Therefore, the cap can be easily removed from the connector by pulling the tab and tearing the strip along the perforations 188 (188′), 288 (288′). Once the strip is torn but may still be attached to the cap, the cap can be easily removed from the connector.

A cap may be formed from any suitable material which provides the cap with sufficient structural integrity and is sufficiently pliable such that the strip 187 (187′), 287 (287′) can be easily torn along the perforations 188 (188′), 288 (288′). Preferably the cap is formed from a plastic material or a metallic material, such as aluminum or aluminum alloy. More preferably the cap is formed from a polymeric material such as polycarbonate or polypropylene.

In accordance with one aspect of the present invention, the connector assembly includes at least one resilient sealing member, such as a male sealing member 270 (270′) disposed at the proximal end of male connector 200 (200′). For example, the male sealing member may be enclosed in a socket 245 (245′) formed on the proximal end of the male connector 200 (200′) and having an open end. In the illustrated embodiment, the socket 245 (245′) is defined by the annular sidewall 244 (244′) at the connecting end of the male connector 200 (200′), and the open end comprises the proximal end surface 243 (243′) of the side wall 244 (244′). The socket 245 (245′) preferably completely surrounds the male sealing member 270 (270′); e.g., the side wall 244 (244′) preferably comprises a continuous, unbroken cylindrical wall which completely surrounds the male sealing member 270 (270′). The socket 245 (245′) and the male sealing layer 310 (310′) preferably sealingly contain the resilient sealing member(s).

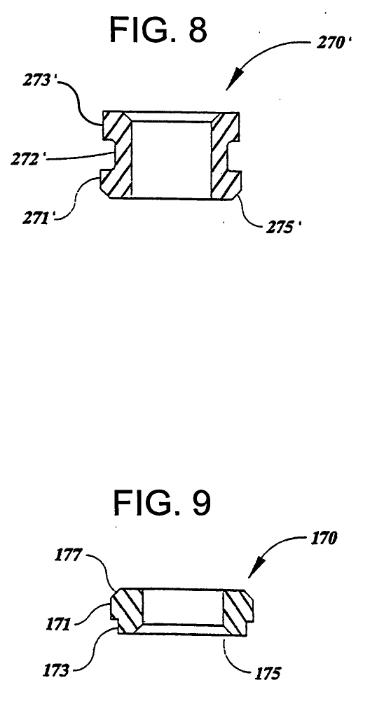

The male sealing member 270 (270′) can be variously configured. For example, the male sealing member 270 (270′) may comprise a resiliently compressible and expandable member including a hollow body having opposite open ends and an interior passage extending between the open ends. In the exemplary embodiment shown in FIG. 8, the male sealing member 270 (270′) preferably comprises an annular base portion 271 (271′), neck portion 272 (272′), and head portion 273 (273′). The base portion 271 (271′) preferably comprises an annular rim having a slightly larger outer diameter than the inner diameter of the sidewall 244 (244′) and being adapted to form a tight frictional fit with the sidewall 244 (244′) when it is inserted in the socket 245 (245′) of the male connector 200 (200′). The base portion 271 (271′) may include a beveled surface 275 (275′) along its outer diameter to allow the base portion 271 (271′) to be inserted in and slide to the bottom of the socket 245 (245′).

The neck portion 272 (272′) of the male sealing member 270 (270′) preferably forms an annular wall joining the base portion 271 (271′) and the head portion 273 (273′). The wall of the neck portion 272 (272′), which is preferably thinner than the wall of the base portion 271 (271′) and thinner than the wall of the head portion 273 (273′), is preferably resiliently compressible to allow the male sealing member 270 (270′) to be compressed within the socket 245 (245′) of the male connector 200 (200′) by the male stripout layer 310 (310′). In the illustrated embodiment, the length of the male sealing member 270 (270′) is greater than the length of the male sidewall 244 (244′) and the thin wall neck portion 272 (272′) has an inner diameter equal to, and an outer diameter less than, those of the base portion 271 (271′) and the head portion 273 (273′). The neck portion 272 (272′) resiliently collapses, e.g., bends radially outwardly, to allow the sealing member 270 (270′) to be compressed within the socket 245 (245′) of the male connector 200 (200′). Alternative structures for the neck portion 272 (272′) are within the scope of the present invention. For example, the neck portion 272 (272′) may have a larger inner diameter than those of the base portion 271 (271′) and head portion 273 (273′) and may bend radially inward, or the neck portion 272 (272′) may comprise a bellows-like member having multiple bends when the male sealing member 270 (270′) is compressed.

The head portion 273 (273′) preferably comprises a beveled inner surface 277 (277′) and an annular rim which is formed on an end of the male sealing member 270 (270′) opposing the base, member 271 (271′). Further, the head portion 273 (273′), as well as the neck portion 272 (272′), preferably has an outer diameter which is smaller than the outer diameter of the base portion 271 (271′) and is smaller than the inner diameter of the side wall 244 (244′) forming the socket 245 (245′). Because the outer diameters of the head portion 273 (273′) and the neck portion 272 (272′) are smaller than the inner diameter of the socket 245 (245′) and are spaced from the side wall 244,(244′) of the socket 245 (245′), they easily expand axially within the socket 245 (245′) without seizing or catching against the side wall 244 (244′). Thus, the head portion 273 (273′) and the neck portion 272 (272′) may resiliently expand from within the socket 245 (245′) to form a tight seal with the female connector when the stripout layers are removed. For example, when male connector 200 is coupled to female connector 100′, the head portion 273 and the neck portion 272 may resiliently expand from within the socket 245 to form a tight seal with the female connector 100′ when the stripout layers 300, 310′ are removed.

There are many alternative ways by which a male sealing member may be configured. For example, a male sealing member can have (not shown) a head portion and a base portion which have substantially the same outer diameter. The socket can have a continuous cylindrical wall including an interior step in which the inner diameter of the distal portion of the socket wall is smaller than that of the proximal portion of the socket wall. Preferably the inner diameter of the distal portion of the socket wall is slightly less than the outer diameter of the base portion and is adapted to form a tight frictional fit with the base portion when the male sealing member is inserted in the socket. The inner diameter of the proximal portion of the socket wall preferably is larger than the outer diameters of the head portion and the neck portion such that the head and the neck portions can easily expand axially within the socket without seizing or catching against the proximal portion of the socket wall.

Although the illustrated embodiment depict the male sealing member 270 (270′), as having a constant inner diameter and a varying outer diameter, a male sealing member with a constant outer diameter and variable inner diameter is within the scope of the invention. As long as the male sealing member is resiliently compressible and expandable, the male sealing member may have a varying inner diameter rather than a varying outer diameter. Alternatively, the male sealing member may have a varying inner diameter and a varying outer diameter or a constant inner diameter and a constant outer diameter.

A second sealing member, for example, a female sealing member 170 (170′), may be disposed in the socket 145 (145′) of the female connector 100 (100′). The socket 145 (145′), which also has an open end, includes the sidewall 144 (144′), which is preferably continuous and completely surrounds the female sealing member 170 (170′), and the proximal end surface 143 (143′) of the female fitting 120 (120′). The female sealing member is preferably sealingly contained within the socket 145 (145′) and the female stripout layer 300 (300′).

The female sealing member 170 (170′) may be variously configured. For example, the female sealing member 170 (170′) may also comprise a resiliently compressible and expandable member including a hollow body having opposite open ends and an interior passage extending between the open ends. The female sealing member 170 (170′) preferably comprises abase portion 171 (171′) and ahead portion 173 (173′), e.g., as shown in FIG. 9. The base portion 171 (171′) preferably comprises an annular rim having an outer diameter larger than the inner diameter of the sidewall 144 (144′) and being adapted to form a tight frictional fit with the socket 145 (145′) of the female connector 100 (100′). The base portion 171 (171′) preferably also includes a beveled outer surface 175 (175′) to facilitate insertion of the female sealing member 170 (170′) into the bottom of the socket 145 (145′).

The head portion 173 (173′), as well as the base portion 171 (171′), preferably comprises a resiliently compressible material to allow the female sealing member 170 (170′) to be compressed within the socket 145 (145′) of the female connector 100 (100′). The head portion preferably has an outer diameter which is smaller than the outer diameter of the base portion 171 (171′) and is smaller than the inner diameter of the side wall 144 (144′) forming the socket 145 (145′). Because the outer diameter of the head portion 173 (173′) is smaller than the inner diameter of the socket 145 (145′) and is spaced from the side wall 144 (144′) of the socket 145 (145′), the head portion easily moves axially within the socket without seizing or catching against the side wall. Thus, the head portion 173 (173′) may resiliently expand within the socket 145 (145′) to form a tight seal with the male connector 200 (200′) when the stripout layers (e.g., 300 and 310′, or 300′ and 310) are removed. The head portion 173 (173′) preferably comprises an inner diameter and a beveled inner surface 177 (177′) which mirror the inner diameter and the beveled inner surface 277 (277′) of the male sealing member 270 (270′) to form an annular indention 163 (163′) in an inner surface of the joined sealing members (e.g., 170 and 270′, or 170′ and 270) when the stripout layers are removed. Further, the head portion 173 (173′) may have a thinner wall than that of the base portion 171 (171′).

There are also many alternative ways by which a female sealing member may be configured. For example, a female sealing member can have (not shown) a uniform outer diameter. The socket can have a continuous cylindrical wall including an interior step in which the inner diameter of the distal portion of the socket wall is smaller than that of the proximal portion of the socket wall. Preferably the inner diameter of the distal portion of the socket wall is slightly less than the outer diameter of the female sealing member and is adapted to form a tight frictional fit with the female sealing member when the female sealing member is inserted in the socket. The inner diameter of the proximal portion of the socket wall preferably is larger than the outer diameter of the female sealing member such that the female sealing member can easily expand axially within the socket without seizing or catching against the proximal portion of the socket wall.

The sealing member or members provide several advantages. For example, each sealing member 170 (170′), 270 (270′) may be formed from a different material than the material forming the fittings 120 (120′), 220 (220′). In particular, each sealing member may be formed from a material which is more resilient, e.g., more resiliently compressible and expandable, than the more rigid material forming the fittings 120 (120′), 220 (220′). Exemplary materials for the sealing members include resiliently compressible and expandable polymeric materials or elastomeric materials. A preferred material is a TPE (thermoplastic elastomer), such as a SANTOPRENE® TPE. The enhanced resiliency of the sealing member(s) provides a greatly improved seal. Another advantage of the sealing member or members is that the end surface of the head portion 173 (173′), 273 (273′) may be formed very evenly, providing an excellent seal. In preferred embodiments, the end surfaces of the head portions 173 (173′), 273 (273′) of the contained sealing members 170 (170′), 270 (270′) abut but are not joined to the stripout layers 300 (300′), 310 (310′), i.e., the stripout layers are joined only to the end surfaces 143 (143′), 243 (243′) of the cylindrical walls 144 (144′), 244 (244′). This allows the end surfaces of the head portions 173 (173′), 273 (273′) to remain even and clean and, thereby, form a tight seal free of any leachants. Of course, in less demanding applications, the stripout layers may be joined to both the sidewalls and the sealing members or only to the sealing members.

Although the illustrated embodiment depicts a female sealing member 170 (170′) being sealed in the socket 145 (145′) of the female connector 100 (100′) by the female stripout layer 300 (300′), and the male sealing member being compressed and sealed within the socket 245 (245′) of the male connector 200 (200′) by the male stripout layer 310 (310′), alternative arrangements are within the scope of the present invention. For example, the male sealing member 270 may be disposed in the socket 145′ of the female connector 100′, and the female sealing member 170 may be disposed in the socket 245′ of the male connector 200′. Alternatively, the female sealing member 170 (170′) may be omitted. In an embodiment in which the female sealing member 170 (170′) is omitted, the male sealing member 270 (270′) may be disposed within the socket of either couplable connector by a stripout layer or a non-removable sealing layer.

In an embodiment which includes a single sealing member, when the stripout layer is removed, the sealing member may abut a surface on the connecting end of the opposing couplable connector to seal the connector assembly. For example, if a male sealing member 270 is disposed in the socket 245 of the male connector 200, the head portion 273 of the connector may contact a surface 135′ in the counterbore 136′ of the female connector 100′. Alternatively, the sidewall 144′ of the female connector may be thickened in a radially inward direction to extend inwardly beyond the sidewall 244 of the male connector and provide a contact surface for the male sealing member 270.

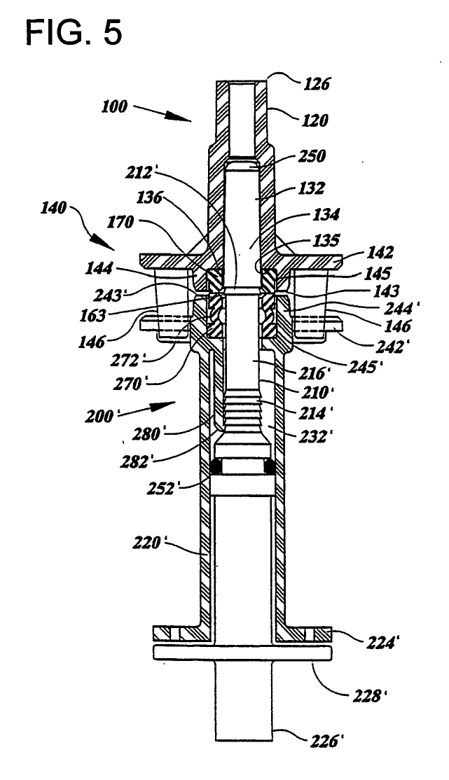

The male connector 200 (200′) preferably includes a stem 210 (210′) telescopically housed in a generally cylindrical body 221 (221′) defining the aperture 232 (232′) in the male fitting 220 (220′), e.g., as shown in FIGS. 4 and 5. The male connector 200 (200′) is also preferably adapted to contain and conduct fluid communication and preferably defines an isolated portion of the fluid flow path, e.g., containing or conducting isolated fluid communication. Accordingly, the stem 210 (210′) is preferably sealed within the aperture 232 (232′) defined by the fitting 220 (220′). In the illustrated embodiment, the stem 210 (210′) includes a seal 252 (252′) coupled between a distal end 226 (226′) of the stem 210 (210′) and the body 221 (221′) of the male connector 200 (200′). The seal 252 (252′) may comprise an o-ring disposed around the stem 210 (210′). In an alternative embodiment, the seal 252 (252′) may be disposed in a groove in the interior wall of the body of the male connector 200 (200′). The seal 252 (252′) preferably sealingly and slidably engages an interior wall to seal the aperture 232 (232′) from the ambient environment and allow the stem 210 (210′) to move axially.

While a stem 210 (210′) of a male connector may be arranged to move axially only with respect to the female connector the male connector will be coupled thereto, and to be stationary with respect to the male fitting 220 (220′), the stem 210 (210′) is preferably arranged to move axially both with respect to the female connector and the male fitting 220 (220′). For example, the stem 210 (210′) preferably moves axially through the male fitting 220 (220′); e.g., through the aperture 232 (232′) and the open proximal end of the aperture 232 (232′), through the socket 245 (245′) and the open end bf the socket 245 (245′), through the male sealing member 270 (270′) including the open ends and the interior passage, and/or through any non-removable sealing layer. Further, the stem 210 (210′) preferably moves axially into the female connector; e.g., stem 210 of connector 200 moves axially through any non-removable sealing layer, through the female sealing member 170′ including the open ends and the interior passage, through the open end of the socket 145′ and the socket 145′, through the open end of the aperture 132′, and/or into the aperture 132′. Because the stem 210 moves through the female and/or male sealing members, the largest outer diameter of the stem 210 is preferably smaller than the smallest inner diameter of the interior passages of the sealing members 170′, 270. Further, the proximal portion of the stem 210 preferably is tapered and has a bullet-shaped configuration. This facilitates axial movement of the stem 210 without disturbing the seal formed by the sealing members 170′, 270. Alternatively, the diameters may be approximately equal to create a seal between the stem 210 and the sealing member or members 170′,270.

The stem 210 (210′) is preferably hollow, defining a lumen (not shown) therein. The proximal end of the stem 210 (210′) may have a head 250 (250′) formed thereon. The head 250 (250′) may have an aperture providing fluid access between the lumen and the exterior of the stem 210 (210′). The head 250 (250′) may comprise a blunt member or a piercing member, depending on whether or not the sealing layers include non-removable layers. For example, if the sealing layers include non-removable layers in addition to stripout layers, the head 250 (250′) preferably comprises a piercing member to pierce the non-removable layers and provide fluid communication between the interior regions of the male and female connectors (e.g., male connector 200 and female connector 100′, or male connector 200′ and female connector 100). If separate non-removable layers are not included, the head 250 (250′) may comprise a blunt member. The head 250 (250′) may be blunt because once the stripout members are removed, there are no obstructions which require piercing between the couplable male and female connectors.

The stem 210 (210′) is preferably connected to a conduit as shown in the Figures. For example, a second conduit 102 (102′) comprising flexible tubing maybe connected to the distal end 226 (226′) of the stem 210 (210′) in any suitable manner, e.g., by using solvents, bonding agents, hose clamps, ultrasonic welding, threaded connectors, or friction fitting. Alternatively, the tubing may be molded integrally with the stem 210 (210′).

According to another aspect of the present invention, a stem 210 (210′) may include a locking device. The locking device 260 (260′) may be of any configuration that restricts the accidental or inadvertent axial advancement of the stem 210 (210′). In the embodiment illustrated in FIG. 3, the locking device comprises two locking tabs 260 (260′) rigidly extending axially from a lower flange 224 (224′) of the body 221 (221′) to a flange 228 (228′) on the stem 210 (210′). The number of locking tabs 260 (260′) is not critical to the invention. For example, a single locking tab 260 (260′) may be included, or more than two locking tabs 260 (260′) may be included. If multiple locking tabs 260 (260′) are included, they are preferably located at equally spaced circumferential locations about the stem 210 (210′) to uniformly distribute force applied to the stem 210 (210′).

In the embodiment shown in FIG. 3, the locking tabs 260 (260′) comprise radially projecting fins which extend axially between the flanges 224 (224′), 228 (228′). The locking tabs 260 (260′) maybe deformable, e.g., maybe arranged to bend out of the way or to break away from one or both of the flanges 224 (224′), 228 (228′). For example, the locking tabs 260 (260′) may be attached at bendable or frangible joints 262 (262′) to the flange 228 (228′) and/or the barrel of the stem 210 (210′). The locking tabs 260 (260′) are preferably not attached to the distal flange 224 (224′) of the male fitting 220 (220′). Thus, each locking tab 260 (260′) may be easily grasped and bent in a direction perpendicular to the plane of the tab 260 (260′), breaking the frangible joint and freeing the stem 210 (210′) to move axially. In an alternative embodiment, the locking device may comprise a permanently attached, non-breakable arrangement, such as a radially extending key on the stem 210 (210′) and a keyway on the body 221 (221′) which allows the axial movement of the key, and stem 210 (210′) after the key is aligned with the keyway. Alternatively, the stem 210 (210′) may include one or more keyways and the body 221 (221′) may include one or more keys.

Typically, the locking device comprises one or more wings extending radially from the surface of the stem 210 (210′), wherein the wings extend radially beyond the inner diameter of the male fitting 220 (220′) and may abut the distal surface of the flange 224 (224′), thus preventing the stem 210 (210′) from being inadvertently advanced within the male fitting 220 (220′). In order to advance the stem 210 (210′), the stem 210 (210′) may be rotated. The rotation of the stem 210 (210′) pushes the wings tangentially against a structure that can apply a tangential force to the wings. As a result, the wings bend tangentially and fold away from the distal surface of the flange 224 (224′), thus allowing the stem 210 (210′) to advance within the male fitting 220 (220′). For example, each of the wings can be disposed within a slot on the distal surface of the flange 224 (224′). When the stem 210 (210′) is rotated, the rotation of the stem 210 (210′) pushes the wings against the sidewalls of the slots and bends the wings tangentially, thus allowing the stem 210 (210′) to advance within the male fitting 220 (220′). Alternatively, the distal surface of the flange may include protrusions instead of slots, and the rotation of the stem pushes the wings against the protrusions and bends the wings tangentially, thus allowing the stem to advance within the male fitting. In a preferred embodiment of the locking device, nothing needs to be broken off and, therefore, there are no loose pieces associated with the locking device.

A purpose of the locking devices is to restrict the accidental or inadvertent axial advancement of the stem 210 (210′). Preferably, an operator does not unlock the locking device until the male connector 200 and the female connector 100′ (or the male connector 200′ and the female connector 100) are joined and the stripout layers (e.g., 300 and 310′) are removed. If the locking device is unlocked before the connectors are joined and the stripout layers are removed, the stem 210 (210′) may damage the stripout layer 300 (300′) and compromise the sterility of the male connector 200 (200′).

In addition to the locking device, the male connector 200 (200′) may also comprise a ratchet structure. For example, as shown in FIGS. 4 and 5, the stem 210 (210′) may comprise first and second sets of beveled annular ribs 212 (212′), 214 (214′) circumfusing the external surface of the stem 210 (210′). The ribs maybe beveled such that they project from the surface of the stem 210 (210′), extending distally toward the flange 228 (228′) of the stem 210 (210′) and forming an acute angle with the external surface of the stem 210 (210′). The first set of ribs 212 (212′) is preferably spaced from the second set of ribs 214 (214′) by a smooth surface 216 (216′) formed on the stem 210 (210′). A catching member 280 (280′) is preferably coupled to the inner wall of the body 221 (221′) of the male connector 200 (200′). A distal end of the catching member 280 (280′) includes a catch 282 (282′) which rests on the outer surface of the stem 210 (210′). A similar ratchet structure is disclosed in U.S. Pat. No. 5,393,101, which is incorporated by reference to support this and other features of the present invention. The ratchet structure can comprise a single set of annular ribs and preferably does not include a smooth surface section. The ratchet structure in U.S. Pat. No. 5,393,101 is preferred in some embodiments because the stem is not retractable once the head is advanced toward the female fitting, and can only move toward the female fitting.

The stem 210 (210′) may further include a device disposed between the male fitting 220 (220′) and the stem 210 (210′), which stabilizes the stem when the stem is advanced within the male fitting. An exemplary embodiment of the device may include a plurality of axially extending ribs. The ribs may be mounted, for example, on the stem 210 (210′) between the O-ring 252 (252′) and the flange 228 (228′) and preferably are equally spaced circumferentially around the stem 210 (210′). The outer surfaces of the ribs may define a cylinder that has a diameter similar to the inner diameter of the male fitting 220 (220′). Thus, when the stem 210 (210′) is advanced within the male fitting 220 (220′), the outer surfaces of the ribs contact the inner surface of the male fitting 220 (220′), which stabilizes the stem 210 (210′) as it moves along within the male fitting 220 (220′).

In operation, to join the connectors (e.g., using female connector 100 and male connector 200′ for reference; female connector 100′ can be joined to male connector 200 in a similar manner), an operator first removes the caps 183, 283′ protecting the proximal ends of the connectors 100, 200′ by pulling the tabs 186, 286′ and tearing the strips 187, 287′ along the perforations 188, 288′. The operator then interlocks the connectors 100 and 200′. In the illustrated embodiments, interlocking the connectors comprises sliding the forks 146 in the female connector 100 into the slots 240′ in the male connector 200′ until the catches 148 abut against the distal surface of the flange 242′. As shown in FIG. 1, the forks 146 may bend slightly as the catches 148 at the ends of the forks 146 move through the slots 240′.

Continuing to use connectors 100 and 200′ for reference, the interlocking mechanism may be configured to ensure that the tabs of the stripout layers 300, 310′ both extend in the same direction when the connectors 100, 200′ are interconnected. For example, the forks 146 and slots 240′ may be arranged in sets such that the forks 146 only engage the slots 240′ when the tabs extend in the same direction. In the illustrated embodiment, one set of forks and slots are closely spaced while the other set of forks and slots are more distantly spaced. The tabs, forks, and slots are all arranged such that the connectors 100 and 200′ will interconnect only when the closely spaced forks engage the closely spaced slots, the distantly spaced forks engage the distantly spaced slots, and the tabs extend in the same direction from the stem.

Once the connectors 100, 200′ are coupled, the stripout layers 300, 310′ are removed, which in the illustrated embodiment places the apertures 132, 232′ of the connectors 100, 200′ in fluid communication, with each other. Any contaminants entrained on the external surfaces of the stripout layers 300, 310′ may be removed with the stripout layers 300, 310′.

As each stripout layer 300, 310′ is removed, one or both of the male and female sealing members 270′, 170, which were compressed in the male and female sockets 245′, 145, expand to contact each other and seal the connectors 100, 200′. The sealing members preferably maintain the seal throughout the process of removing the stripout layers 300, 310′. More particularly, as the stripout layers are withdrawn, the exposed portions of the sealing members 170, 270′ expand and contact one another, creating a seal between the contacting exposed portions. Because contact between the sealing members follows the withdrawing stripout layers, the seal is immediately created behind the stripout layers 300, 310′ as the stripout layers are withdrawn.

To contact the female sealing member 170, the resiliently compressible head portion 273′ and/or neck portion 272′ of the male sealing member 270′ axially expands from a compressed state to an expanded state where the distance between the base 271′ and head 273′ portions is increased. The head portion 173 of the female sealing member 170 may also expand. The head portion 273′ of the male sealing member 270′ abuts against the head portion 173 of the female sealing member 170 to form the seal. Because the male sealing member 270′ and the female sealing member 170 each comprise a resiliently compressible and expandable member, movement of the male connector 200′ or the female connector 100 once they are coupled does not reduce the seal. The male and female sealing members 270′, 170 expand or compress to counteract any movement of the connectors 100, 200′ and tightly maintain the seal. The annular groove 163 may decrease the surface area of the contact between the sealing members and thus increase the axial pressure exerted on one sealing member by the other, thereby strengthening the seal. Thus, a tight, contaminant-free connection is created and maintained.

Once the stripout layers 300, 310′ are removed, the head 250′ of the stem 210′ is preferably extended into the female connector 100. In order to move the head axially, an operator unlocks the locking device, for example, by grasping and breaking the locking tabs 260′ away from the flange 228′ of the stem 210′ in the case of the embodiment shown in FIG. 1, or, in the embodiment including wings, by rotating the stem 210′ to deform the wings tangentially. The operator then slides the flange 228′ of the stem 210′ axially towards the lower flange 224′ of the male connector 200′. As the stem moves axially, the stem 210′, including the head 250′, moves through the male fitting 220′ and the female connector 100 as previously described. Further, the seal 252′ slides along the inner wall of the male connector 200′; the catching member 280′ slides along the first ribbed surface 212′ and the smooth surface 216′ and then latches along the second ribbed surface 214′; and the head 250′ then lodges in the bore 134 of the female connector 100. The bore 134 is preferably tapered so the head 250′ lodges in frictional sealing engagement with the wall of the bore 134. Fluid (e.g., cell culture medium, more typically, cells suspended in cell culture medium) may then flow freely without contamination from the container of one cell culture device to the container of the other cell culture device through the aperture 132 in the female connector 100 and the lumen in the stem 250′ via the contaminant-free connection of the female and male connectors 100, 200′.

In other embodiments (not shown), a female connector may be connected directly to a container, e.g., through a fluid flow port. For example, the female connector may be fitted with a fitment such as a transfer leg closure. The female connector and the container may be constructed as a single, integral unit. Alternatively, or additionally, a male connector may be connected directly to a container.

As noted above, embodiments of systems according to the invention can include any number of cell culture devices, and one cell culture device can differ from another with respect to at least one of any of the following: the number of connectors, the types of connectors, the connector elements, the number of ports, the type of ports, the gas permeability of the walls of the containers, the polymeric film used in manufacturing the containers, and the size of the containers.

Moreover, embodiments of the cell culture system according to the invention can include one or more additional components, such as, for example, a filter, a filter device, a vent, as well as additional conduits, containers, one or more connectors, and one or more flow control devices such as clamps, transfer leg closures, and valves.

In some embodiments, cells (and/or cell populations) of interest can be harvested, concentrated and/or selected before and/or after placing cells in cell culture devices according to the invention. For example, cells of interest can be harvested, concentrated and/or selected as disclosed in U.S. Pat. No. 6,544,751, and the cells can subsequently processed in the inventive cell culture device.

In an embodiment, the cell culture device and/or cell, culture system includes a system for use in determining the presence of microorganisms (e.g., bacteria) in the cell-containing fluid. This can be especially desirable for those embodiments wherein the cultured cells are to be administered to a patient, e.g., to minimize the potential that microorganism-contaminated cell-containing fluid is administered to a patient. In one embodiment, the system for use in determining the presence of microorganisms includes a filter, e.g., the filter disclosed in International Publication No. WO 01/32828.

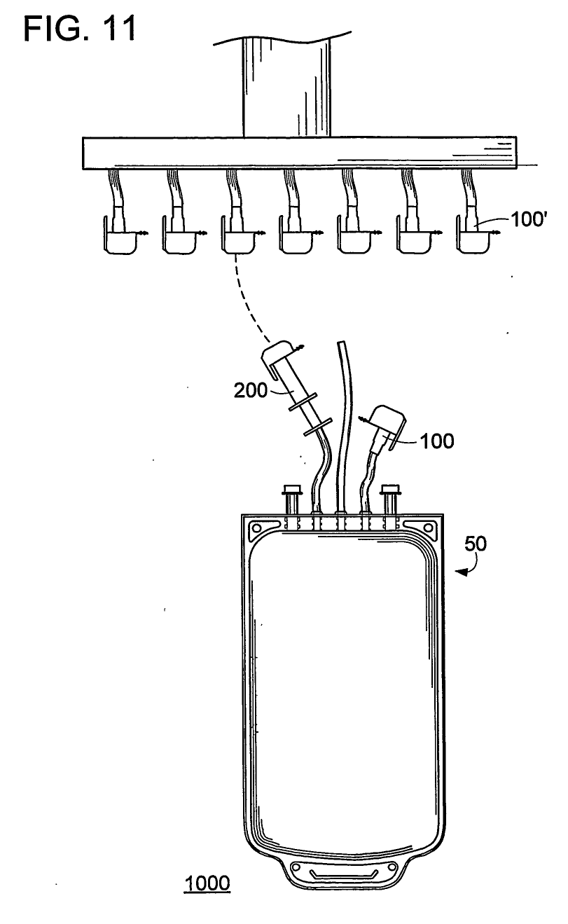

If desired, embodiments of cell culture devices according to the invention can be coupled to other types of arrangements, systems and/or devices to allow fluid to be transferred into and/or out of the cell culture device. For example, FIG. 11 shows the embodiment of the cell culture device illustrated in FIG. 1 (including an illustrative male connector 200 and an illustrative female connector 100), as well as a partial view of a manifold having a plurality of female connectors 100′, wherein the male connector 200 can be coupled to a manifold female connector 100′ to allow fluid to be passed into the cell culture device and/or from the cell culture device. Another portion of the manifold (not shown) can communicate with, for example, a source of cell culture medium, a source of cells, or a container for harvested cells. The source or container can comprise, but is not limited to, for example, another cell culture device, or a bioreactor of any suitable volume. If desired, a clamp (not shown) can be utilized with each conduit communicating with a connector used during fluid transfer, and the clamps can be opened and closed as appropriate. The connectors can be coupled to provide an aseptic connection as described above. While the illustrated manifold shows a plurality of female connectors, the manifold can include any combination of connectors, e.g., a plurality of male connectors and/or female connectors.

In some embodiments including additional containers, at least one of the additional containers need not have gas permeable walls, e.g., it is not used for culturing cells. Accordingly, additional containers can be made from conventional polymers and/or copolymers as is known in the art.

The following example further illustrates the invention but, of course, should not be construed as in any way limiting its scope.

### EXAMPLE

This example shows cells can be maintained in culture for 31 consecutive days and sub-cultured 4 times without contamination, using devices and systems prepared in accordance with embodiments of the invention.

A series of bags are prepared from an ethylene butyl acrylate copolymer (EBAC) resin, EBAC SP1802 (22.5 wt. % butyl acrylate comonomer) as generally described in International Publication No. WO 02/065976. Each bag has a plurality of ports. In forming each cell culture device, a first port is connected to a first flexible conduit having a first fitting at the end of the conduit, a second port is connected to a second flexible conduit having a second fitting at the end of the conduit, and a third port is connected to a third flexible conduit having a sealed end (for use in sterile docking). Each device includes first and second fittings so that a first fitting attached to one device can be coupled to a second fitting attached to another device.

Connector assemblies are prepared as generally described in International Publication No. 98/50105 and attached to the bags via flexible conduits, wherein a first fitting (hereinafter the “female connector”), connected via flexible tubing to one bag, is couplable to a second fitting (hereinafter the “male connector”), connected via flexible tubing to another bag. The male and female connectors each define an aperture and include a resilient rubber grommet coupled to the connector at the aperture, and each connector also includes a removable vented 0.2 micron bacteria blocking sealing layer sealing the aperture. Each connector has a removable cap.

The male connector includes a stem member (plunger) mounted in the fitting, the plunger including a head axially movable into the aperture of the female connector after the male and female connectors coupled. The resilient rubber grommet for the male connector includes a hub and a neck joined to the hub.

Each device, i.e., a bag with capped male and female connectors, is sterilized via gamma sterilization.

FALCON™ Flasks (Becton-Dickenson) are also obtained.

Promyeloblast cells (HL-60: ATCC#CCL240) are grown in 5% CO₂ at 37° C. in Iscove's modified Dulbecco's medium containing L-glutamine and 20% fetal bovine serum. Cells (3×10⁴/mL) are introduced into the source bags and the flasks.

When the cells reach the plateau phase of the growth curve, the source bags are connected to transfer bags through the connector assemblies and the passage of cells is accomplished in room air without using a laminar flow hood.

In making the connection, the caps covering the male and female connectors attached to the respective bags are moved. The male and female connectors are snapped together and are resiliently coupled in antagonistic based-opposition. The removable sealing layers are pulled out simultaneously via pull tabs. The plunger is moved axially so that the head moves into the aperture of the female connector, and flow is initiated.

About 10% of the contents of the source bag is passed to the transfer bag (the transfer bag containing fresh cell culture medium), the conduits communicating with the transfer bag and the source bag are heat sealed.

Cells are also transferred from the source flasks to the transfer flasks. While under a laminar flow hood, the caps on the flasks are removed, the cells are transferred via pipettes, and the caps are replaced on the flasks.

The cell density is monitored, and the cells are transferred when the cells reach the plateau phase of the growth curve. The cells are passed, four times, to new bags and flasks, as described above.

The cells in the last set of transfer bags and flasks are analyzed on day 32.

Analysis of the cells in the bags shows no contamination, and the results show the proliferation index (viable cell concentration/initial viable cell concentration), cell density, and cell viability using the bags is comparable to that using the flasks.

This example shows, using devices according to embodiments of the invention, the cells can be cultured and sub-cultured in the devices, and one device can be connected to another, while maintaining a contaminant-free fluid pathway. The example also shows device-based cell culture methods are consistent with Good Manufacturing Processes, and are comparable to those of flask-based culture methods.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A cell culturing device comprising:
a first container having an internal volume and at least first and second fluid flow ports, the container having first and second side walls permeable to gas, the first and second side walls each comprising a polymeric film having a 22° C. room air oxygen transmission of about 12 μmoles or greater O₂/hr/350 cm² film surface area; and,
a first fitting in fluid communication with the internal volume of the container, the first fitting including a first removable sealing layer, being couplable to an additional fitting for use in providing a contaminant-free connection, the additional fitting being in fluid communication with an additional container.

2. The cell culturing device of claim 1, further comprising a first conduit having a first end and a second end, wherein the first end is connected to the first fluid flow port of the container, and wherein the second end is connected to the first fitting.

3. The cell culturing device of claim 1, further comprising:
a second fitting in fluid communication with the internal volume of the first container, the second fitting being couplable to an additional fitting for use in providing a contaminant-free connection, the additional fitting being in fluid communication with a fluid flow port of an additional container.

4. The cell culturing device of claim 3, further comprising a second conduit having a first end and a second end, wherein the first end is connected to the second fluid flow port of the container, and wherein the second end is connected to the second fitting.

5. The cell culturing device of claim 1, wherein the walls of the container comprise a polymeric film manufactured from a copolymer comprising ethylene and an alkyl acrylate, the alkyl acrylate comprising butyl acrylate or methyl acrylate.

6. The cell culturing device of claim 1, wherein the walls of the container comprise a polymeric film manufactured from plasticized PVC.

7. (canceled)

8. The cell culturing device of claim 3, wherein the second fitting includes a second removable sealing layer.

9. The cell culturing device of claim 1, wherein the container includes at least a third fluid flow port.

10. (canceled)

11. A cell culturing system comprising:
a first cell culturing device according to claim 1; and,
a second cell culturing device comprising a second container having an internal volume and at least first and second fluid flow ports, the container having first and second side walls permeable to gas; and, at least a second fitting in fluid communication with the internal volume of the container;
wherein the first fitting of the first cell culturing device is couplable with the second fitting of the second cell culturing device to provide a contaminant-free connection.

12. The cell culture system of claim 11, including a first conduit having a first end and a second end, the first end attached to the first fitting of the first cell culturing device, and the second end attached to the first fluid flow port of the first cell culturing device; and
including a second conduit having a first end and a second end, the first end attached to the second fitting of the second cell culturing device, and the second end attached to the second fluid flow port of the second cell culturing device.

13. A cell culturing system comprising:
a first cell culturing device comprising a first container having an internal volume and at least first and second fluid flow ports, the first container having first and second side walls permeable to gas; and first and second fittings in fluid communication with the internal volume of the first container, the first and second fitting of the first cell culturing device each defining an aperture and including a removable sealing layer sealing the aperture; and,
a second cell culturing device comprising a second container having an internal volume and at least first and second fluid flow ports, the second container having first and second side walls permeable to gas; and first and second fittings in fluid communication with the internal volume of the second container, the first and second fitting of the second cell culturing device each defining an aperture and including a removable sealing layer sealing the aperture;
wherein at least the first fitting of the first cell culturing device is couplable to the second fitting of the second cell culturing device.

14. The cell culture system of claim 13, wherein the second fitting of the first cell culturing device is couplable to the first fitting of the second cell culturing device.

15. The cell culture system of claim 13, wherein first fitting of the first culture device includes a stem member mounted in the fitting, the stem member having a head axially movable into the aperture of the second fitting of the second cell culture device.

16. The cell culture system of claim 14, wherein first fitting of the second culture device includes a stem member mounted in the fitting, the stem member having a head axially movable into the aperture of the second fitting of the first cell culture device.

17. A cell culturing system comprising:
a first container having an internal volume and at least first and second fluid flow ports, the first container being manufactured from a gas permeable film and having first and second side walls permeable to gas;
a first portion of a connector assembly, the first portion of the connector assembly comprising a first fitting, the first fitting defining a first aperture, and including a first resilient sealing member coupled to the first aperture, the first fitting including a first removable sealing layer sealing the first aperture, wherein the first fitting is in fluid communication with the internal volume of the first container; and,
a second container having an internal volume and at least first and second fluid flow ports, the second container being manufactured from a gas permeable film and having first and second side walls permeable to gas;
a second portion of the connector assembly, the second portion of the connector assembly comprising a second fitting, the second fitting defining a second aperture, and including a second resilient sealing member coupled to the second aperture, the second fitting including a second removable sealing layer sealing the second aperture, wherein the second fitting is in fluid communication with the internal volume of the second container;
wherein the first fitting is couplable with the second fitting.

18. The cell culture system of claim 11, wherein the walls of the first and/or second container comprise a polymeric film manufactured from a copolymer comprising ethylene and an alkyl acrylate, the alkyl acrylate comprising butyl acrylate or methyl acrylate.

19. The cell culture system of claim 11, wherein the walls of the first and/or second container comprise a polymeric film manufactured from plasticized PVC.

20. The cell culture system of claim 17, wherein the polymeric film is manufactured from a copolymer comprising ethylene and at least about 18 weight percent alkyl acrylate, the alkyl acrylate comprising butyl acrylate or methyl acrylate.

21. A method for processing cells comprising:
maintaining cells in a cell culture medium in a first cell culturing device according to claim 1.

22. The method of claim 21, further comprising coupling the first fitting of the first cell culturing device to a fitting of an additional cell culturing device, and passing the cells from the first cell culturing device to the additional cell culturing device.

23. The method of claim 22, further comprising maintaining the cells in a cell culture medium in the additional cell culturing device.

24. A method for processing cells comprising:
coupling the first fitting of the first cell culturing device to the second fitting of the second cell culturing device of the system of claim 11;
establishing a contaminant-free connection between the first fitting and the second fitting; and,
passing a cell- and cell medium-containing fluid from the first cell culturing device to the second cell culturing device.
